(19) United States
(12) Patent Application Publication
(10) Pub. No.: US 2025/0352051 A1
(43) Pub. Date: Nov. 20, 2025

(54) DENTAL TREATMENT SYSTEM AND METHOD USING AI TECHNOLOGY

(71) Applicant: UA ROBOTICS CO., LTD., Seongnam-si (KR)

(72) Inventors: Ki Woong LIM, Seoul (KR); Shinwook KANK, Seoul (KR)

(21) Appl. No.: 18/854,554

(22) PCT Filed: May 31, 2022
(86) PCT No.: PCT/KR2022/007727

§ 371 Date: Oct. 7, 2024

(30) Foreign Application Priority Data
Apr. 7, 2022 (KR) 10-2022-0043246

(51) Int. Cl. A61B 1/247

(52) U.S. Cl. CPC A61B 1/247

(57) ABSTRACT

The present invention relates to a dental treatment system and method using AI technology, the dental treatment system comprising: a CBCT imaging unit which photographs the positions of the jawbone on the inside of the teeth and the teeth of a patient; a camera which selects a treatment tool used for a robot arm, tooth milling, and an implant procedure, and is installed on the selected treatment tool to obtain tooth and gum areas of the patient in real time; and an AI server which, on the basis of images photographed in advance by the CBCT imaging unit and images obtained in real time through the camera during treatment, recognizes when the treatment tool is close to a tooth and gum area to be treated, and according to a recognition result, determines whether to continue to proceed with treatment or give a warning.

### TECHNICAL FIELD

The present invention relates to a dental treatment system and method using an AI technology, more specifically to a dental treatment system and method using an AI technology that is capable of allowing a camera to be mounted on a robot arm or handpiece used in a dental clinic so that an image acquired in real time through the camera and an image captured through a CBCT imaging unit are analyzed using the AI technology, and next, if the robot arm or handpiece escapes from a tooth and gum area to be treated, a caution is given with a warning sound or lamp to allow surgery to be performed in a safer and more precise manner.

### BACKGROUND ART

As computerized tomography (CT) and image scanning have been recently commercialized in dental treatments such as teeth milling, implant surgery, prosthetic treatment, and the like, a dentist can monitor the structure of the jawbone on images, without any invasive incision. Further, as captured CT images and scanned images are reconstructed three dimensionally and provided for the dentist, effective planning before surgery can be built, and an improved technology such as guidance during surgery can be provided.

While surgery is being actually performed by the dentist, however, it is impossible to utilize the CT images and the scanned images, and therefore, if the teeth have a bad arrangement or are not good in position, the surgery may not succeed. Otherwise, other teeth may be badly influenced by the tooth treated during the surgery, and further, nerves near the tooth treated during the surgery may be undesirably damaged.

Further, CBCT imaging is widely utilized in dental treatments because through it, the jawbone inside the teeth and the positions of the teeth can be accurately recognized, but since radiation harmful to the human body is used for the CBCT imaging, the dentist may be excessively exposed to radiation, making it difficult to perform long surgeries while monitoring CBCT images in real time.

To solve such problems, there is a need to develop a dental treatment system and method that is capable of utilizing information of CT and image scanning, while surgery is being performed by a dentist, to allow the surgery to be performed accurately according to planning before the surgery built by the dentist.

### DISCLOSURE OF THE INVENTION

### Technical Problems

Accordingly, the present invention has been made in view of the above-mentioned problems occurring in the related art, and it is an object of the present invention to provide a dental treatment system and method using an AI technology that is capable of allowing a camera to be mounted on a robot arm or handpiece used in a dental clinic so that an image acquired in real time through the camera and an image captured through a CBCT imaging unit are analyzed using the AI technology, and next, if the robot arm or handpiece escapes from a tooth and gum area to be treated, a caution is given with a warning sound or lamp to allow surgery to be performed in a safer and more precise manner.

It is another object of the present invention to provide a dental treatment system and method using an AI technology that is capable of allowing a camera to be mounted on a handpiece used directly by a dentist so that if other teeth, not a tooth to be treated are erroneously treated by the dentist, a caution is given to the dentist, thereby allowing surgery to be performed safely.

It is yet another object of the present invention to provide a dental treatment system and method using an AI technology that is capable of allowing a camera to be mounted on a robot arm of a dental robot so that surgery is performed in a safer and more precise manner.

### Technical Solutions

To accomplish the above-mentioned objects, according to an aspect of the present invention, there is provided a dental treatment system using an AI technology, the dental treatment system including: a CBCT imaging unit for capturing images of the jawbone inside the teeth of a patient and the positions of the teeth; a camera mounted on a surgical tool such as a robot arm and a tool used for teeth milling or implant surgery to capture in real time images of the tooth and gum area of the patient; and an AI server based on the images captured by the CBCT imaging unit and the image captured in real time through the camera during surgery to recognize the surgical tool closing to the tooth and gum area to be treated so that it is determined whether the surgery is kept performed or a warning is given according to the recognized result.

The camera mounted on the surgical tool may be any one selected from a mini camera and a 3D camera having a laser sensor or IR sensor and capable of distance recognition.

The surgical tool may be a handpiece used directly by a dentist, the camera being mounted on the handpiece, and the surgical tool may be a dental robot, the camera being mounted on the robot arm of the dental robot.

To accomplish the above-mentioned objects, according to another aspect of the present invention, there is provided a dental treatment method using an AI technology, the dental treatment method including the steps of: capturing images of the jawbone inside the teeth of a patient and the positions of the teeth through a CBCT imaging unit; selecting a tooth and gum area to be treated in an image guidance program based on the images captured through the CBCT imaging unit; automatically acquiring an image having a similar angle to the 2D image captured in real time through a camera among the 3D reconstructed images based on the images captured through the CBCT imaging unit, if surgical planning is made by a dentist; if the acquisition of the image has been completed, performing AI analysis for 3D target coordinates, an entry angle, and an entry depth of the tooth and gum area to be treated and the selected image and transmitting the analyzed result to an AI server; and performing the surgery for the patient, while receiving in real time the information of the tooth and gum area to be treated from the AI server.

The step of performing the surgery may include the steps of: performing the surgery through a handpiece used directly by a dentist; capturing the image of the tooth and gum area in real time through the camera mounted on the handpiece and transmitting the captured image and the distance value between the tooth and the gum to the AI server; if the transmission of the image and the distance value has been completed, recognizing whether the tooth and gum area selected on the image selected from the images captured through the CBCT imaging unit is the same as the image captured in real time through the camera or recognizing some teeth, if teeth rarely exist or a recognition rate is low, to estimate the selected tooth and gum area using center distance values of the teeth or through recognition of a specific object fixed in the mouth; and if the handpiece used by the dentist is close to a given range of the selected tooth and gum area, normally performing the surgery, and if the handpiece escapes from the given range, giving a caution to the dentist.

In the step of giving the caution, if the handpiece may escape from the given range during the direct surgery of the dentist, the caution may be given with a warning lamp or sound.

The step of performing the surgery may include the steps of: performing the surgery using the camera mounted on a robot arm of a dental robot; capturing the image of the tooth and gum area in real time through the camera mounted on the robot arm of the dental robot and transmitting the captured image and the distance value between the tooth and the gum to the AI server; if the transmission of the image and the distance value has been completed, recognizing whether the tooth and gum area selected on the image selected from the images captured through the CBCT imaging unit is the same as the image captured in real time through the camera or recognizing some teeth, if teeth rarely exist or a recognition rate is low, to estimate the selected tooth and gum area using center distance values of the teeth or through recognition of a specific object fixed in the mouth; and if the dental robot is close to a given range of the selected tooth and gum area, normally performing the surgery, and if the dental robot escapes from the given range, giving a caution.

In the step of giving the caution, if the dental robot may escape from the given range during the surgery through the dental robot, the surgery may be temporarily stopped, and the caution may be given with a warning sound.

### Advantageous Effects of the Invention

According to the present invention, the dental treatment system and method is configured to allow the camera to be mounted on the robot arm or handpiece used in a dental clinic. Therefore, the image acquired in real time through the camera and the image captured through the CBCT imaging unit are analyzed using the AI technology, and next, if the robot arm or handpiece escapes from the tooth and gum area to be treated, a caution is given with a warning sound or lamp, so that the surgery is performed in a safer and more precise manner.

### BRIEF DESCRIPTION OF DRAWINGS

FIG. 1 is a block diagram showing a basic configuration of a dental treatment system according to the present invention.

FIG. 2 is a block diagram showing a connection configuration of the dental treatment system according to the present invention to a dental robot and a handpiece.

FIG. 3 shows an image captured by a CBCT imaging unit according to the present invention.

FIGS. 4a and 4b show planning before surgery built using a 3D reconstruction program and selected CBCT images according to the present invention.

FIG. 5 shows the CBCT images automatically selected through the dental treatment system before surgery to enhance a degree of recognition accuracy in an AI technology according to the present invention.

FIG. 6 is a perspective view showing a state where a camera is mounted on the handpiece according to the present invention.

FIG. 7 is a perspective view showing a state where a camera is mounted on a robot arm according to the present invention.

FIGS. 8, 8a, 8b, 8c, and 8d show AI teeth recognition states through the camera according to the present invention.

FIG. 9 is a block diagram showing the dental treatment system using the AI technology according to the present invention.

FIG. 10 is a flowchart showing shows a dental treatment method using an AI technology according to the present invention.

### MODE FOR INVENTION

The present invention may be modified in various ways and may have several exemplary embodiments. Specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention. If it is determined that the detailed explanation on the well-known technology related to the present invention makes the scope of the present invention not clear, the explanation will be avoided for the brevity of the description.

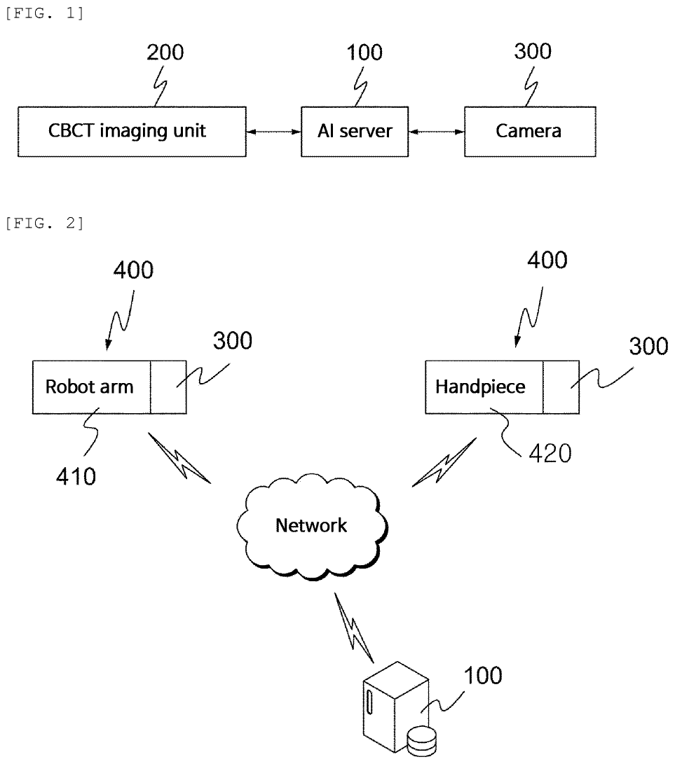

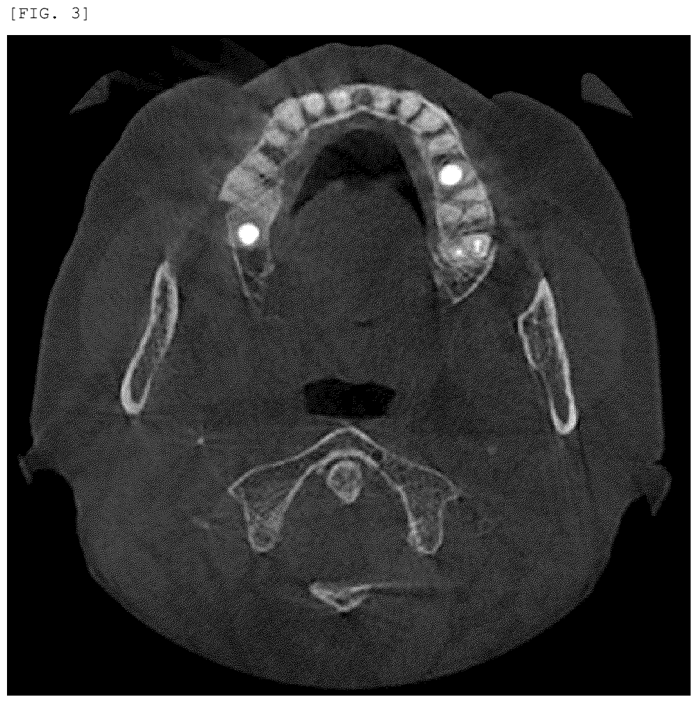

FIG. 1 is a block diagram showing a basic configuration of a dental treatment system according to the present invention, and FIG. 2 is a block diagram showing a connection configuration of the dental treatment system according to the present invention to a dental robot and a handpiece.

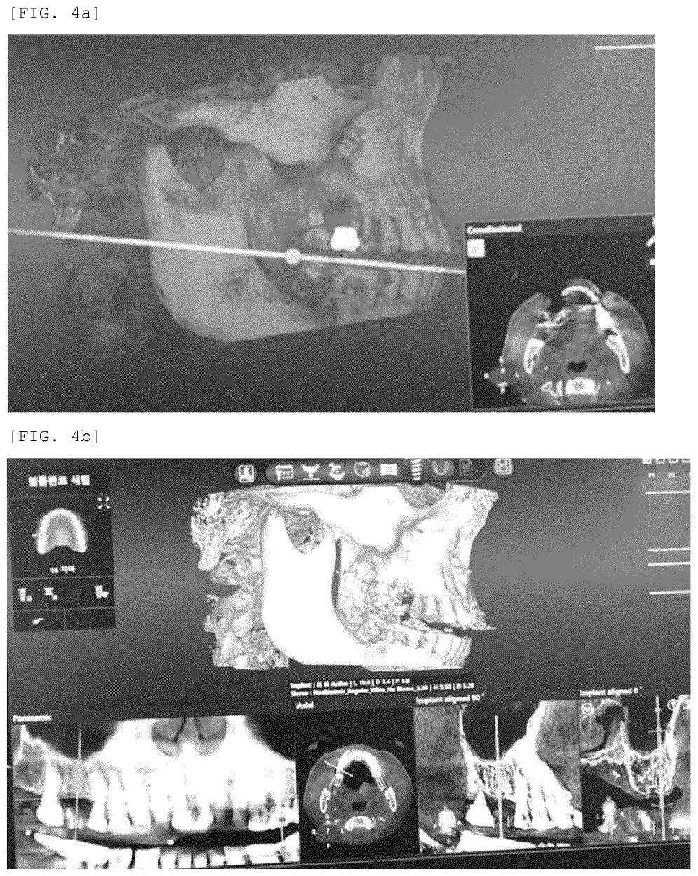

FIG. 3 shows an image captured by a CBCT imaging unit according to the present invention, FIGS. 4a and 4b show planning before surgery built using a 3D reconstruction program and selected CBCT images according to the present invention, and FIG. 5 shows the CBCT images automatically selected through the dental treatment system before surgery to enhance a degree of recognition accuracy in an AI technology according to the present invention.

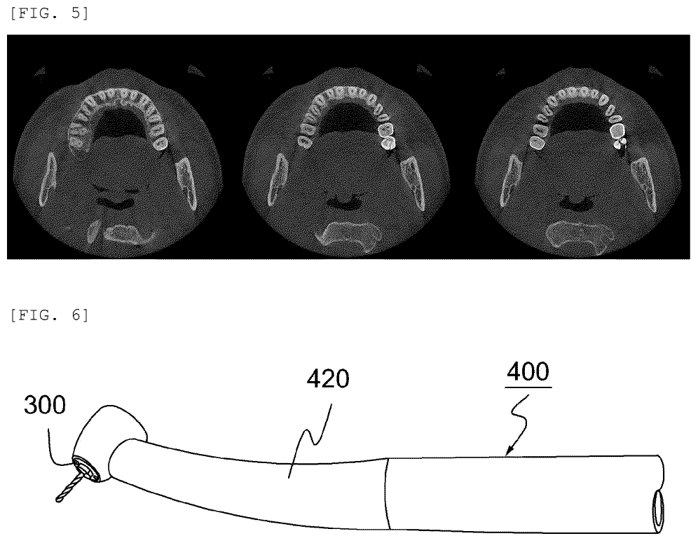

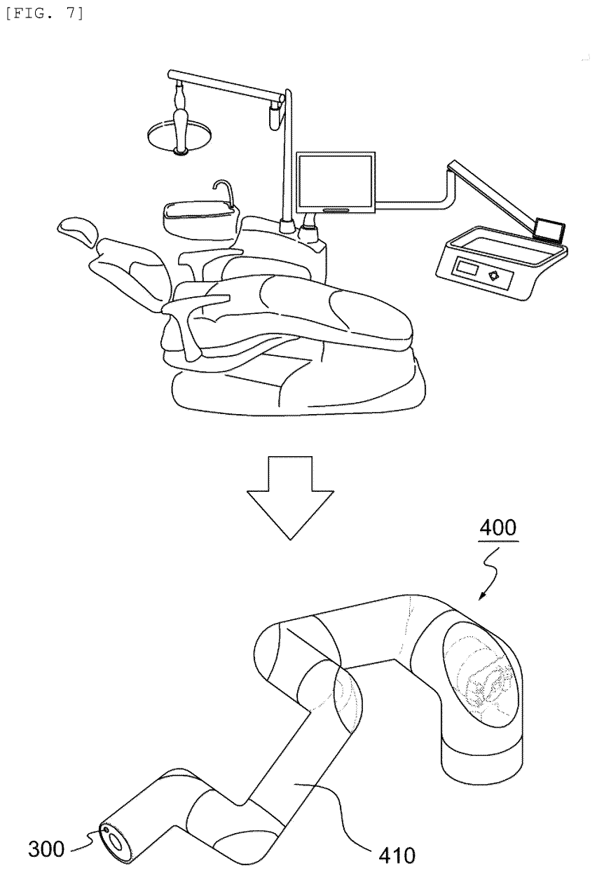

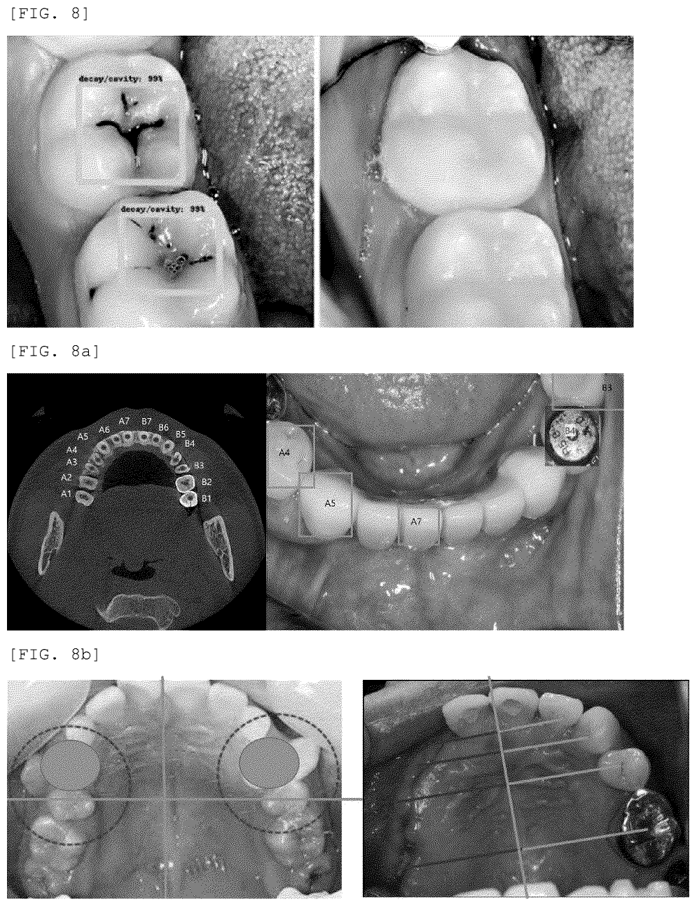

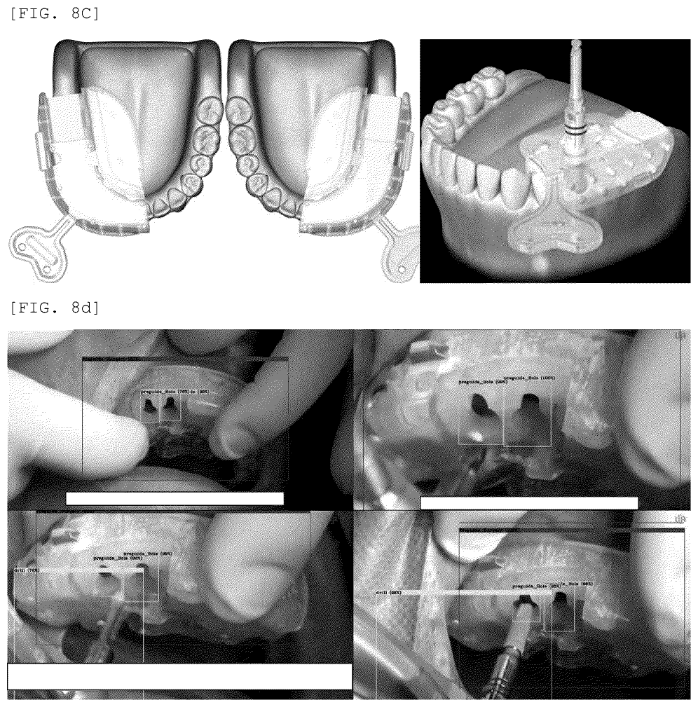

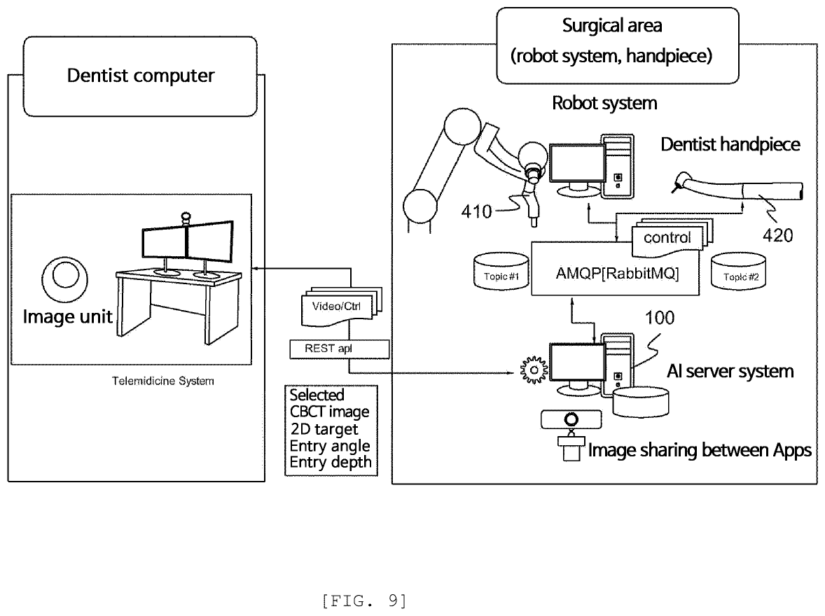

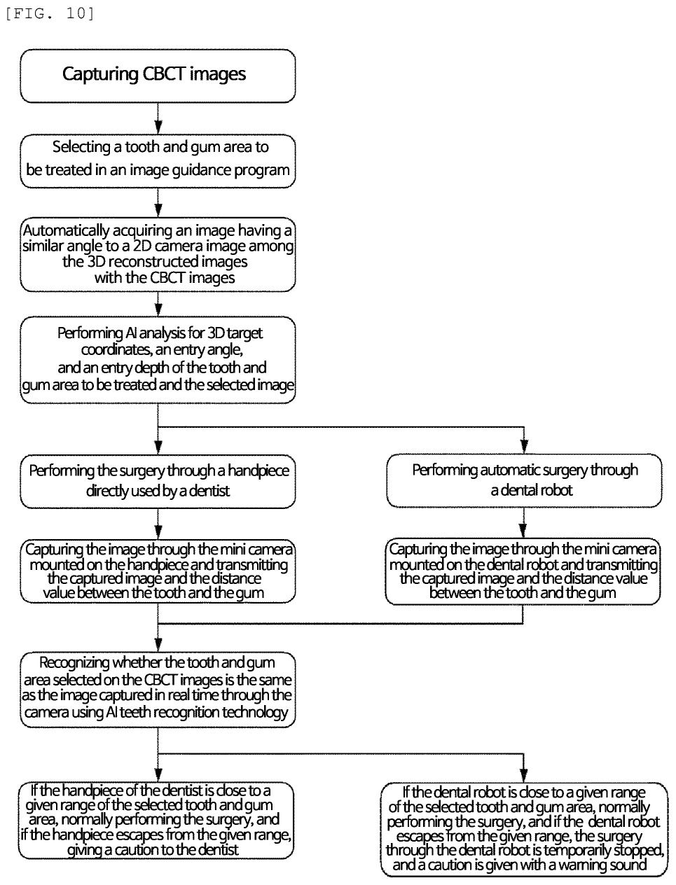

FIG. 6 is a perspective view showing a state where a camera is mounted on the handpiece according to the present invention, FIG. 7 is a perspective view showing a state where a camera is mounted on a robot arm according to the present invention, FIGS. 8, 8a, 8b, 8c, and 8d show AI teeth recognition states through the camera according to the present invention, FIG. 9 is a block diagram showing the dental treatment system using the AI technology according to the present invention, and FIG. 10 is a flowchart showing shows a dental treatment method using an AI technology according to the present invention.

As shown in FIGS. 1 and 2, a robot arm 410 connected to a dental robot and a handpiece 400 used directly by a dentist, which are used to accomplish the objects of the present invention, will be already known and used in the art, and therefore, an explanation of the robot arm 410 and the handpiece 400 will be avoided for the brevity of the description. A term ‘surgical tool 400’ as will be discussed later represents the robot arm 410 connected to a dental robot and the handpiece 400 used directly by a dentist.

As shown in FIGS. 1 and 2, a dental treatment system according to the present invention basically includes a CBCT imaging unit 200, a camera 300, and an AI server 100.

The cone-beam computed tomography (CBCT) imaging unit 200 is generally used for implant surgery, determination of maxillary sinus floor elevation diagnosis and surgery, treatments of impacted wisdom tooth, supernumerary tooth, and temporomandibular joint, osteoclasia of periodontal disease, apicoectomy, and orthognathic surgery estimation, and the CBCT imaging unit 200 captures images of the jawbone inside the teeth of a patient and the positions of the teeth.

The image data captured through the CBCT imaging unit 200 is reconstructed as a 3D panorama image. That is, a DICOM file obtained through CBCT imaging is transmitted to a computer, and next, a CBCT-reconstructed panorama image such as panoramic radiographs is taken using a 3D image program.

If a position and an angle for surgery are determined using the 3D image program by a dentist, as shown in FIGS. 4a and 4b, the CBCT image corresponding to the selected tooth or gum surface is automatically selected around an axis and displayed on a lower position of the image, and the CBCT image is transmitted to the AI server 100 so that it is compared with a real-time camera image, thereby enabling tooth recognition. If the dentist feels that the image automatically selected in the program is not accurate, as shown in FIG. 5, he or she monitors the pre-captured and post-captured images of the automatically selected image and thus selects another CBCT image, and the selected image is transmitted to the AI server 100.

To use the camera 300, the surgical tool 400 used for teeth milling and implant surgery is selected, and next, the camera 300 is mounted on the selected surgical tool 400 to capture the tooth and gum area of the patient in real time.

The camera 300 mounted on the surgical tool 400 is any one selected from a mini camera and a 3D camera having a laser sensor or IR sensor and capable of distance recognition, and the mini camera or 3D camera as the camera 300 is appropriately chosen, depending upon the situations.

As shown in FIG. 6, the surgical tool 400 may be the handpiece 420 used directly by the dentist, and in this case, the camera 300 is mounted on the handpiece 420. Further, as shown in FIG. 7, the surgical tool 400 may be a dental robot, and in this case, the camera 300 is mounted on the robot arm 410 of the dental robot. This allows the objects of the present invention to be accomplished when the surgery is performed directly by the dentist and when the surgery is performed by the dental robot.

It is desirable that the camera 300 be generally mounted in the same direction as a drill, but only if the camera 300 captures an image in real time to achieves its object, it may be mounted on a module connecting the robot arm 410 and the handpiece 420.

Further, the position and area of the tooth and gum planned before the surgery can be recognized through the application of an AI tooth recognition technology to the image selected in the 3D imaging program among the images captured through the CBCT imaging unit 200 and to the image captured in real time through the camera 300 or through the AI recognition of an object made by a 3D printer for accurate surgery.

In detail, the tooth selected on the image captured through the CBCT imaging unit 200 is accurately displayed accurately because it is a hard material including the jawbone and gum, and since the tooth is an S/W image reconstructed three-dimensionally, it is possible to acquire images for all areas around the tooth. Further, teeth have numbers from the center of the mouth to the back, and the selected tooth number is transmitted to the AI server 100, together with the tooth image, so that it is possible to recognize the selected tooth or gum through the recognition of teeth and gum around the selected tooth.

The image captured through the camera 300 attached to the robot arm 410 or the handpiece 420 is a 2D image that may be obtained when teeth are seen at upper and lower positions of the mouth in the state where the mouth is open. Therefore, the image having a similar angle to the 2D camera image is automatically obtained among the images reconstructed three dimensionally with the CBCT images.

This increases recognition rates in the AI recognition technology and image segmentation technology since the image having a similar angle to the 2D camera image is automatically obtained among the images reconstructed three dimensionally with the CBCT images in consideration of the angle of the mouth opened of the patient for the dental surgery.

To recognize a position of a surgical area having a low recognition rate, further, some teeth, which are not the surgical area selected by the dentist, are recognized and relatively estimated through a distance value relation. To do this, as shown in FIG. 8a, 3D coordinate values x, y, and z of each tooth center on the CBCT images are automatically or manually acquired in the 3D image program, and the magnification of an RGB camera on the CBCT image is calculated, so that even though some teeth are recognized through a closed-loop algorithm using the distance values of the teeth, the surgical area as desired by the dentist can be estimated.

Further, as shown in FIG. 8b, teeth and gums are symmetrical, and using such symmetricity, some teeth, not the surgical area, are recognized to allow the surgical area to be estimated.

Furthermore, as shown in FIG. 8c, in the case of recent dental implant surgery, a surgical area is determined in the 3D image program, and if it corresponds to the patient's teeth, a transparent plastic object having a hole formed correspondingly to the surgical area is accurately made for an individual patient through 3D printing, so that a pre-guide and the hole are recognized using the AI technology, thereby allowing the position of the tooth to be treated to be recognized as shown in FIG. 8d.

Therefore, if the AI teeth recognition technology is utilized, it can be recognized whether the tooth and gum area selected from the CBCT image is the same as the image captured in real time through the camera 300. Like this, the tooth is recognized through the AI teeth recognition technology, and further, the tooth selected in the CBCT image is compared with the shape of the tooth acquired through the image segmentation technology, thereby making it possible to the recognize the tooth to be treated.

In the case where the patient's tooth is already removed or some teeth do not exist in the dental treatment such as implant surgery, through the image segmentation technology as well as the teeth recognition technology as mentioned above, the similarity in the shape of the gum to the CBCT image is obtained, and using feature points (recognition of molars located on both ends of the mouth) having high similarity probability, it can be recognized that 3D target coordinates, an entry angle, and an entry depth received through a network correspond to the selected tooth or gum area through relative position calculation. Further, teeth have numbers from the center of the mouth to the back, and the selected tooth number is transmitted to the AI server 100, together with the tooth image, so that it is possible to recognize the selected tooth or gum through the recognition of teeth and gum around the selected tooth.

The AI server 100, which is based on the image already captured through the CBCT imaging unit 200 and the image captured in real time through the camera 300 during the surgery, recognizes the surgical tool 400 that is close to the tooth and gum area to be treated and determines whether the surgery is kept performed or a warning is given according to the recognized result.

### Embodiment

The dental treatment system and method using the AI technology according to the present invention is provided, as shown in FIGS. 9 and 10.

Image capturing step;

Images of the jawbone inside the teeth of a patient and the positions of the teeth are captured through the CBCT imaging unit 200, and the image data captured through the CBCT imaging unit 200 is reconstructed as the 3D panoramic image through the 3D reconstruction program.

Surgical area selection step;

A tooth and gum area to be treated is selected in an image guidance program based on the images captured through the CBCT imaging unit 200.

Image acquisition step;

The image having a similar angle to the 2D image captured in real time through the camera 300 is automatically acquired among the 3D reconstructed images based on the images captured through the CBCT imaging unit 200.

Transmission step;

If the acquisition of the image has been completed in the image acquisition step, the AI analysis for the 3D target coordinates, the entry angle, and the entry depth of the tooth and gum area to be treated and the selected image is performed, and the analyzed result is transmitted to the AI server 100.

Surgical step;

If the transmission of the selected image and the information of the tooth to be treated to the AI server 100 has been completed, the surgery for the patient is performed. In this case, a surgical method is determined so that the surgery is performed directly by the dentist or automatically the dental robot.

### In the Case of the Surgery by the Dentist

Dentist surgery step;

The surgery is performed through the handpiece 420 used directly by the dentist.

Distance value transmission step;

The tooth and gum area is captured in real time through the camera 300 mounted on the handpiece 420, and the captured image and the distance value between the tooth and the gum are transmitted to the AI server 100.

Recognition step;

If the transmission of the distance value has been completed, it is recognized whether the tooth and gum area selected on the image selected from the images captured through the CBCT imaging unit 200 is the same as the image captured in real time through the camera 300.

Warning step;

If the handpiece of the dentist is close to a given range of the selected tooth and gum area, the surgery is normally performed, and if it escapes from the given range, a caution is given to the dentist. In this case, if the handpiece escapes from the given range in the warning step, the caution is given with a warning lamp or sound, and after the caution has been removed, next, the surgery is kept performed.

### In the Case of the Automatic Surgery by the Dental Robot

Dental robot surgery step;

The surgery is performed using the camera 300 mounted on the robot arm 410 of the dental robot.

Distance value transmission step;

The tooth and gum area is captured in real time through the camera 300 mounted on the robot arm 410 of the dental robot, and the captured image and the distance value between the tooth and the gum are transmitted to the AI server 100.

Recognition step;

If the transmission of the distance value has been completed, it is recognized whether the tooth and gum area selected on the image selected from the images captured through the CBCT imaging unit 200 is the same as the image captured in real time through the camera 300.

Warning step;

If the dental robot is close to a given range of the selected tooth and gum area, the surgery is normally performed, and if it escapes from the given range, that is, the surgical area, a caution is given. In this case, if the dental robot escapes from the given range in the warning step, the surgery through the dental robot is temporarily stopped, and the caution is given with a warning sound, so that after the caution has been removed, the surgery is kept performed.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings. It is therefore intended that the scope of the invention be limited not by this detailed description, but rather by the claims appended hereto.

[Explanations of reference numerals]

| | |
|---|---|
| 100: AI server | 200: CBCT imaging unit |
| 300: Camera | 400: Surgical tool |
| 410: Robot arm | 420: Handpiece |

### INDUSTRIAL APPLICABILITY

The camera is mounted on the robot arm or handpiece used in a dental clinic. The image acquired in real time through the camera and the image captured through the CBCT imaging unit are analyzed using the AI technology, and next, if the robot arm or handpiece escapes from the tooth and gum area to be treated, a caution is given with a warning sound or lamp, so that the surgery is performed in a safer and more precise manner.

1. A dental treatment system using an AI technology, comprising:
a CBCT imaging unit for capturing images of the jawbone inside the teeth of a patient and the positions of the teeth;
a camera mounted on a surgical tool such as a robot arm and a tool used for teeth milling or implant surgery to capture in real time an image of a tooth and gum area of the patient; and
an AI server configured to recognize the surgical tool approaching to the tooth and gum area to be treated based on the images captured by the CBCT imaging unit and the image captured in real time through the camera during surgery, and to determine whether to continue the surgery or issue a warning according to the recognized result.

2. The dental treatment system according to claim 1, wherein the camera mounted on the surgical tool is any one selected from either a mini camera or a 3D camera equipped with a laser sensor or an IR sensor capable of distance recognition.

3. The dental treatment system according to claim 1, wherein the surgical tool is a handpiece used directly by a dentist, and the camera is mounted on the handpiece.

4. The dental treatment system according to claim 1, wherein the surgical tool is a dental robot, and the camera is mounted on the robot arm of the dental robot.

5. A dental treatment method using an AI technology, comprising the steps of:
capturing images of the jawbone inside the teeth of a patient and the positions of the teeth through a CBCT imaging unit;
selecting a tooth and gum area to be treated in an image guidance program based on the images captured through the CBCT imaging unit;
automatically acquiring an image having a similar angle to the 2D image captured in real time through a camera among the 3D reconstructed images based on the images captured through the CBCT imaging unit, if surgical planning is made by a dentist;
if the acquisition of the image has been completed, performing AI analysis for 3D target coordinates, an entry angle, and an entry depth of the tooth and gum area to be treated and the selected image and transmitting the analyzed result to an AI server; and
performing the surgery for the patient, while receiving in real time the information of the tooth and gum area to be treated from the AI server.

6. The dental treatment method according to claim 4, wherein the step of performing the surgery comprises the steps of:
performing the surgery through a handpiece used directly by a dentist;
capturing the image of the tooth and gum area in real time through the camera mounted on the handpiece and transmitting the captured image and the distance value between the tooth and the gum to the AI server;
after the transmission of the image and distance values is completed, recognizing whether the selected teeth and gum areas from the image captured by the CBCT imaging unit match with the teeth and gums in the real-time image from the camera, or recognizing some teeth, for cases where there are few teeth or low recognition rate, to estimate the selected tooth and gum area using center distance values of the teeth or through recognition of a specific object fixed in the mouth; and
approving the procedure and allowing it to proceed if the handpiece used by the dentist is within a given range of the selected tooth and gum area, and issuing a caution if the handpiece moves outside the given range.

7. The dental treatment method according to claim 6, wherein in the step of giving the caution, if the handpiece escapes from the given range during the direct surgery of the dentist, the caution is given with a warning lamp or sound.

8. The dental treatment method according to claim 5, wherein the step of performing the surgery comprises the steps of:
performing the surgery using the camera mounted on a robot arm of a dental robot;
capturing the image of the tooth and gum area in real time through the camera mounted on the robot arm of the dental robot and transmitting the captured image and the distance value between the tooth and the gum to the AI server;
after the transmission of the image and distance values is completed, recognizing whether the selected teeth and gum areas from the image captured by the CBCT imaging unit match with the teeth and gums in the real-time image from the camera, or recognizing some teeth, for cases where there are few teeth or low recognition rate, to estimate the selected tooth and gum area using center distance values of each teeth or through recognition of a specific object fixed in the mouth; and
if the dental robot is close to a given range of the selected tooth and gum area, normally performing the surgery, and if the dental robot escapes from the given range, giving a caution.

9. The dental treatment method according to claim 8, wherein in the step of giving the caution, if the dental robot escapes from the given range during the surgery through the dental robot, the surgery is temporarily stopped, and the caution is given with a warning sound.
